(19) United States
(12) Patent Application Publication
(10) Pub. No.: US 2011/0059048 A1
(43) Pub. Date: Mar. 10, 2011

(54) THE FUNGUS FUSARIUM SOLANI STRAIN 'FS-K' AND ITS USE IN THE BIOLOGICAL CONTROL OF PLANT PATHOGENS AND IN THE ENHANCEMENT OF PLANT GROWTH AND PRODUCTIVITY

(76) Inventors: Kalliope Papadopoulou, Larissa (GR); Nektarios Kavroulakis, Chania (GR); Spyridon Ntougias, Xanthi (GR); Georgios Zervakis, Alimos (GR); Constantinos Ehaliotis, Athens (GR)

(21) Appl. No.: 12/677,486

(22) PCT Filed: Oct. 9, 2008
(86) PCT No.: PCT/GB2008/000057

§ 371 Date: Apr. 5, 2010

(30) Foreign Application Priority Data
Sep. 11, 2007 (GB) 20070100563

(51) Int. Cl. A01N 63/04; C12N 1/14; A01P 3/00; A01P 1/00; A01P 5/00

(52) U.S. Cl. 424/93.5; 435/256.5

(57) ABSTRACT

This invention refers to the pure culture of a wild isolate of the fungal species Fusarium solani (code name: strain ‘Fs-K’) and to its use for the biological control of plant pathogens. The invention includes the mycelium of the strain ‘Fs-K’ and any genetic modification of it, the asexually produced conidia and chlamydospores of the strain ‘Fs-K’, and the cultures of the strain ‘Fs-K’ (grown on liquid or solid media) which contain the mycelium and/or the conidia and/or the chlamydospores, as well as any extract or ingredient which derives from the growth of the strain ‘Fs-K’ or from the growth of any genetic modification of it. The invention further includes organic substrates for plant growth, as well as any other substrate compositions suitable for the growth of fungi and/or plants which contain forms of the strain ‘Fs-K’ (i.e. any vegetative or reproductive structure of it which is involved in the biological control of diseases). The invention further includes the use of the strain ‘Fs-K’ for the enhancement of plant health and growth, for the protection of plants from pathogenic organisms, and for the regulation of the rhizosphere surroundings.

### DESCRIPTION OF THE INVENTION

The adverse consequences of the irrational application of chemical compounds (pesticides) for the control of plant diseases are noticeable both on the natural environment and the human health. To avoid the excessive use of synthetic inputs in agriculture, novel biological control agents are being developed for protection against plant pathogens; these antagonistic microorganisms confer to plant disease control by suppressing disease incidence and/or by reducing symptoms severity caused by the pathogens.

Composting is the biological decomposition of organic wastes and agro-industrial by-products under controlled aerobic conditions to a relatively stable humus-like material called compost. Composts are widely used as soil conditioners and plant growth substrates, and their ability to suppress the growth of plant pests and pathogens (including bacteria, fungi and nematodes) has been repeatedly demonstrated. These suppressive properties are usually attributed to the antagonistic action of the indigenous compost microbiota against the disease-causing agents.

During the last years a large number of bacteria, yeasts and filamentous fungi were isolated from composts, identified and characterized as regards their suppressive effects against plant pathogens. Various types of biological processes have been proposed for explaining such effects: competition for nutrients and ecological niches, production of cell-wall hydrolytic enzymes and/or of antifungal compounds, parasitism of pathogens, induction of plant defense mechanisms, masking of signals which enhance pathogens growth, etc.

The present invention describes the isolation of a Fusarium solani fungal strain encoded as “Fs-K”, which demonstrates an intense and pronounced antagonistic action (under in vitro conditions) against plant pathogenic fungi and Oomycetes, and with the ability to protect tomato plants from the soil-borne pathogen Fusarium oxysporum f.sp. radicis-lycopersici and from the leaf pathogen Septoria lycopersici. Despite the fact that other Fusarium solani strains presenting suppressive effects against plant pathogens were also recorded in the past, no strain has ever been reported to provide simultaneous protection from both plant pathogens (F. oxysporum f.sp. radicis-lycopersici and S. lycopersici), which are characterized by different modes of invading the host-plant and by different life habits. In addition, the “Fs-K” strain is an endophyte growing into the plant vascular system, and it is effective at the extremely low ratio of inoculum concentration (conidia) 1:10³ of antagonist:pathogen. Moreover, the “Fs-K” strain survives for long time periods (minimum one month) in the plant growth substrates, causing thus a satisfactory suppression effect against soil-borne plant pathogens.

### BRIEF DESCRIPTION OF DRAWINGS (FIGURES)

FIG. 1: Mycelium (a) and conidia (b) of the Fusarium solani strain Fs-K referred in the invention.

FIG. 2: Light micrographs of tomato root tissue at 15 d post-inoculation with Fusarium solani strain Fs-K using Chlorazol Black staining. (A) Intracellular growth of mycelium hyphae (arrows) and formation of round bodies (arrowheads), (B) Development of mycelium hyphae into the plant vascular bundle.

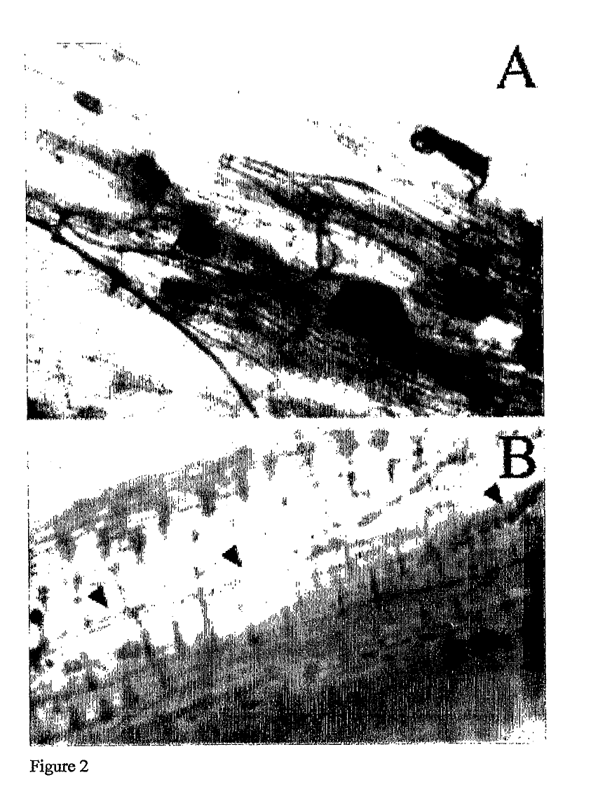

FIG. 3: Disease incidence in tomato plants caused by inoculation with 10⁵ conidia cm⁻³ of Fusarium oxysporum f.sp. radicis-lycopersici. (A) Disease progress curves in tomato plants grown on peat in the absence (triangles) or presence (diamonds) of conidia (10⁴ cm⁻³) of Fusarium solani strain Fs-K referred in the invention. Disease incidence was assessed as percentage of dead plants (five pots, each containing five plants per treatment, n=5). Mean values that were statistically different at each time point (t test P≦0.01) are indicated by an asterisk (*). Vertical bars (I) indicate standard errors of means. (B) Disease incidence caused by Fusarium oxysporum f.sp. radicis-lycopersici in three different tomato cultivars (ACE, VFN8, Pearson) in the absence (−) or presence (+) of 10⁴ conidia cm⁻³ of peat of Fusarium solani strain Fs-K. Statistical comparisons within all cultivars were performed by Duncan multiple comparisons tests (a <0.05). Indicator letters in common denote a lack of significant difference. Vertical bars indicate the ½ of the respective standard errors of means.

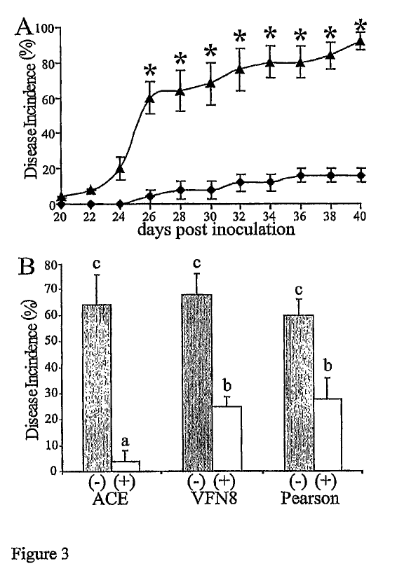

FIG. 4: Effect of Fusarium solani strain Fs-K presence on the development of symptoms caused by Septoria lycopersici strain NEV on tomato plants grown for 1 month on peat (control) or on peat amended with 10⁴ conidia cm⁻³ of Fusarium solani strain Fs-K. Number of lesions was counted per leaf 1 week after inoculation with 10⁶ conidia cm⁻³ of S. lycopersici spore suspension. Vertical bars (I) indicate standard deviations of means (n=12). Statistical comparisons were performed by Duncan's multiple comparisons tests (a <0.005). Letters in common indicate a lack of significant differences.

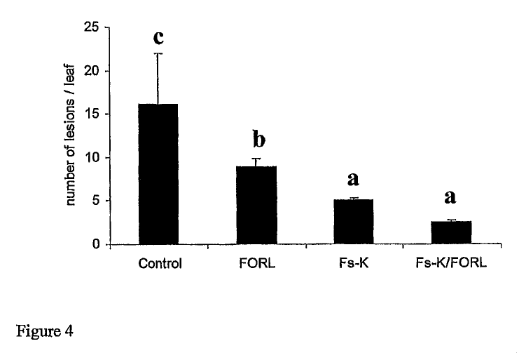

FIG. 5: Linear radial growth of plant pathogens (A) Fusarium oxysporum f.sp. radicis-lycopersici and (B) Phytophthora nicotianae in in vitro tests of the suppressive action of Fusarium solani strain Fs-K culture filtrate. The culture filtrate, after the mycelium removal, was sterilized and added into a PDA growth medium in the ratios (v/v) indicated on the graphs. Statistical comparisons were performed by Duncan's multiple comparisons tests (a <0.005) (n=5). Letters in common indicate a lack of significant difference. Vertical bars (I) indicate the respective standard deviations of means.

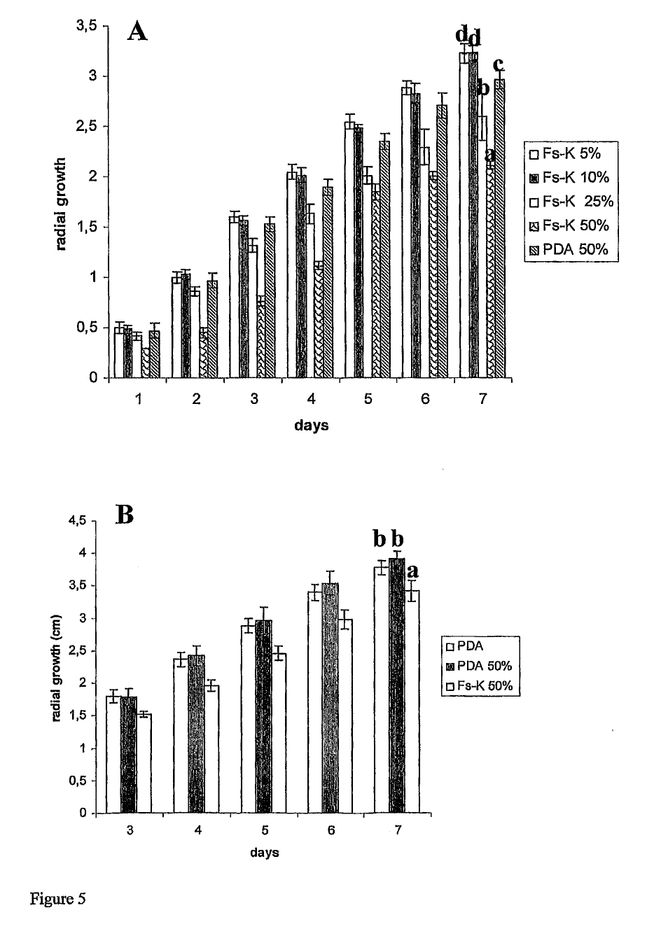

FIG. 6: Increase of the suppressive effect of a compost derived from spent Agaricus mushroom substrate (SMC) against soil-borne plant pathogens through the addition of the Fusarium solani strain Fs-K. The graph describes the percentage of disease incidence symptoms in tomato plants after their infection with 10⁵ conidia cm⁻³ of the plant pathogen Fusarium oxysporum f.sp. radicis-lycopersici. Plants were grown on peat or in SMC compost in the absence or presence (10⁴ [symbol] cm⁻³) of the Fusarium solani strain Fs-K. Disease incidence was assessed as the percentage of dead plants (five pots, each containing five plants per treatment, n=5). Statistical comparisons were performed by Duncan's multiple comparisons tests (a <0.005). Letters in common indicate a lack of significant difference. Vertical bars (I) indicate the respective standard deviations of means.

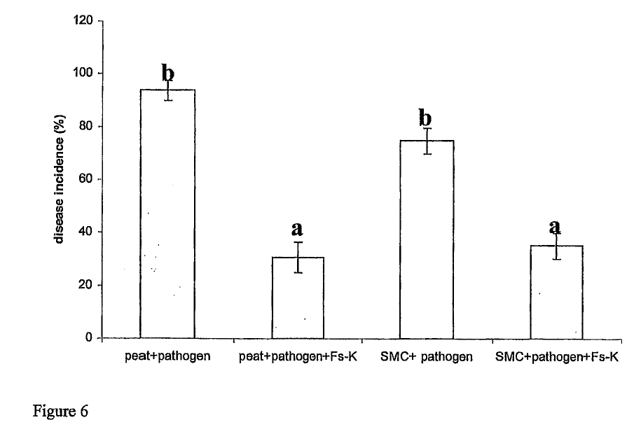

### DETAILED DESCRIPTION OF THE INVENTION

1. Isolation and characterization of Fusarium solani strain Fs-K Fusarium solani strain Fs-K, an antagonistic fungus, was isolated from the roots of tomato plants (line ACE55) which were cultivated in a peat-compost mixture showing suppressive effects against plant pathogenic fungi. The above compost was produced by agricultural residues derived from the winery and olive-oil extraction processes, in particular grape marc (GM, pressed grape pomace) and extracted olive press cake (EPC), in the facilities of the Institute of Olive and Vegetable Crops (National Agricultural Research Foundation-N.AG.RE.F.) which is located in Kalamata, Greece (Kavroulakis et al., 2005).

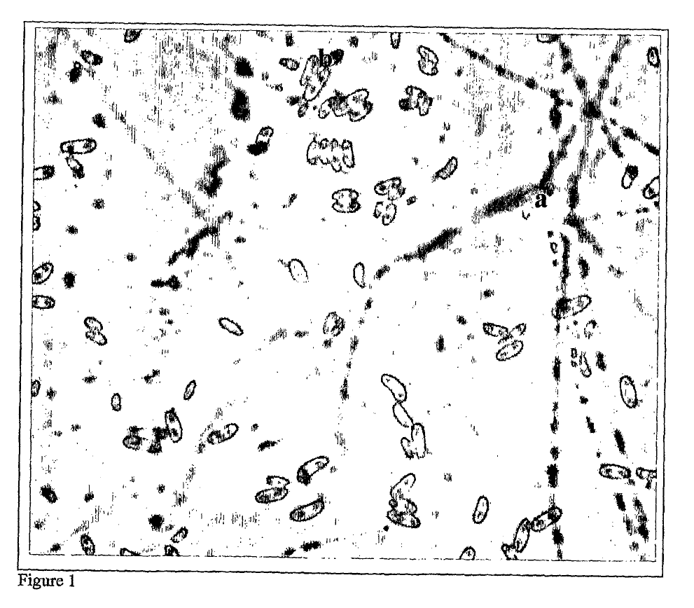

Tomato seeds (line ACE 55) were sterilized with 2.5% NaOCl and sown directly in pots containing peat (Sunu Kura, Seda joint-Stock company) and compost (in a ratio of 1:1 dry wt/dry wt). One month after sowing, roots of tomato plants were surface-sterilized in 2.5% NaOCl for 10 minutes and washed with sterilized tap water. The roots were then placed in Petri dishes containing Potato Dextrose Agar (PDA) medium in which 60 μg ml⁻¹ chlorotetracycline, 30 μg ml⁻¹ streptomycin και 30 U penicillin G were added (Zuberer, 1994) and incubated for approximately 4 days at 25° C. Intraradical mycelial hyphae were subsequently post-inoculated in new sterile liquid and solid PDA media. The fungus, described in the present invention, is filamentous with pale-white hyphae and produces asexual conidia (FIG. 1). The fungal strain described in the invention was isolated from a single conidium (spore). This fungus can be cultivated in several growth media, utilizing a broad range of carbon sources. Its optimum pH and temperature for growth are 5.0 and 24° C. respectively.

This fungal isolate was characterized as Fusarium solani strain based on ITS-5.8S rRNA gene sequencing following the experimental procedure described below: DNA from the fungal strain described in the invention was extracted as described by Lee and Taylor (1990). The Internal Transcribed Spacer 1 (ITS1)-5.8S rRNA gene-Internal Transcribed Spacer 2 (ITS2) DNA region was amplified by polymerase chain reaction (PCR) using primers ITS1 (5′-TCC GTA GGT GAA CCT GCG G-3′) and ITS4 (5′-TCC TCC GCT TAT TGA TAT GC-3′) (White et al., 1990). PCR amplification was performed using a denaturation step of 4 min at 94° C., followed by 35 cycles of 30 sec denaturation at 94° C., 30 sec primer annealing at 52° C. and 1 min DNA chain extension at 72° C. Amplification was completed by 10 min DNA chain extension at 72° C. PCR was performed using a PTC-200 thermocycler (MJ Research Inc., USA). PCR products were separated on 1% agarose gels in 1×TAE buffer, extracted using QIAquick gel extraction columns (Qiagen, Hilden, Germany), cloned into the pGEM-T easy vector (Promega, WI, USA) and sequenced. Similarity searches against sequences deposited in nucleotide data bases were carried out using the Blast-N algorithm.
2. Bioassay for the assessment of the suppressive effects of the fungus described in the present invention against the plant pathogenic fungus Fusarium oxysporum f.sp. radicis-lycopersici

Tomato seeds of different genotypes (ACE 55, Pearson, VFN8, Castlemart) were surface-sterilized in 2.5% NaOCl and sown directly in pots of 15 cm diameter which contained approximately 300 cm³ peat supplemented with a NPK fertilizer (20-20-20) at a final concentration of 0.8 g l⁻¹. The pots were placed in a growth chamber which operated at 20°-25° C. with a photoperiod of 16 h and humidity of 65% RH. Plants were irrigated at 48 h intervals and supplemented weekly with a nutrient solution containing micronutrients.

Plants were routinely inoculated with Fusarium solani strain Fs-K at a concentration of 10⁴ conidia per cm³ peat applied one week after sowing. At the stage of the first true leaf emergence, plants were infected with 10⁵ conidia of the pathogenic fungus Fusarium oxysporum f.sp. radicis-lycopersici per cm³ peat. Control plants were inoculated and infected with a 0.85% NaCl solution only. Five or eight replicates were used for each experimental treatment (replicate pots, each containing five plants).

Conidia of the antagonistic fungus and the pathogenic fungus were derived from the following procedure: The fungi were cultured in potato dextrose broth (PDB) liquid medium. Removal of mycelial fragments by sieving was subsequently performed and conidia were recovered by centrifugation at 4000 g, washed with 0.85% NaCl, counted using a haemocytometer and re-suspended in the appropriate volume of 0.85% NaCl in order to achieve the desired inoculum concentration.

Plants were scored either as healthy (lack of disease symptoms or scarce disease symptoms) or as diseased (severe wilting or dead plants). Tomato plants inoculated with the fungus described in the present invention the antagonist) were statistically significantly more tolerant to the disease caused by the phytopathogenic fungus F. oxysporum f. sp. radicis-lycopersici compared to plants which were cultivated in the absence of the antagonist. All lines and hybrids tested in this bioassay were protected by the fungus described in the invention.

Plants inoculated with the fungus which is described in the present invention did not develop disease symptoms. Observations were performed following the experimental setup up to the stage of fruit development. The antagonistic fungus can grow endophytically in plants, as it was shown for tomato plants. The fungus was isolated from root and crown tissue but never from the upper parts of the plant. Furthermore, the fungus is able to grow in the vascular system of the plant,
3. Effects of inoculum concentration (numbers of conidia per cm³ of growth substrate) of the antagonistic fungus Fusarium solani strain Fs-K on the suppressiveness against the plant pathogen Fusarium oxysporum f.sp. radicis-lycopersici

To examine and determine the inoculum concentration which is required to obtain the disease suppression caused by the antagonistic fungus Fusarium solani Fs-K, the bioassay procedure described in the section 2 was followed for testing a range of conidia concentrations of the fungus described in the present invention. Specifically, tomato plants (line ACE55) were inoculated with 10², 10³, 10⁴, 10⁵ and 10⁶ conidia per cm³ peat. The plant pathogen was added after 7 days at a concentration of 10⁵ conidia per cm³ peat. It was found that even in the lowest antagonistic:pathogen ratio tested (1:10³), the fungus described in the invention showed great suppressiveness against the pathogen, resulting in disease reduction by at least 50% (Table 1).
4. Bioassay for the assessment of suppressive effects of the fungus described in the present invention against the plant pathogenic fungus Septoria lycopersici

Tomato seeds of the line ACE55 were sown and grown in pots containing peat in the presence or absence of the antagonistic fungus described in present invention, as described in section 2. Three replicates (pots, each containing 5 plants) per treatment were used. Twelve detached leaves (four from each replication) were inoculated by brushing the leaf underside with spore suspensions of S. lycopersici NEV [1×10⁶ spores cm⁻³ in 0.01% (v/v) Tween 80] as described by Martin-Hernandez et al. (2000). The underside of control detached leaves was covered with 0.01% (v/v) Tween 80. Five days after inoculation, infection was evaluated by counting the number of necrotic lesions, which are characteristic disease symptoms caused by the pathogen. Leaves from plants inoculated with the antagonistic fungus described in the present invention showed statistically significantly lower numbers of lesions. Thus, the antagonistic fungus described in the present invention also protects plants, preferably tomato plants, from foliar pathogens.
5. In vitro test of inhibitory effects of broth extract (supernatant) from the liquid culture of fungus Fusarium solani Fs-K against the pathogenic micro-organisms Fusarium oxysporum f.sp. radicis-lycopersici and Phytophthora nicotianae

Conidia (10⁵ ml⁻¹) of the fungus Fusarium solani Fs-K were inoculated in PDB and incubated for 5 days at 25° C. and 150 rpm. The grown mycelium was removed by filtration and the supernatant, containing compounds secreted by the fungus, was sterilized. The filtered and sterilized growth medium was used in concentrations 5, 10, 25 και 50% per volume in PDA in order to prepare Petri dishes. The plates were then inoculated with the pathogens Fusarium oxysporum f.sp. radicis lycoperisci and Phytophthora nicotianae. Radial mycelial growth was determined within a 5 days incubation period and compared to growth in control plates in the absence of the filtered growth medium from the antagonistic fungus. Great inhibition in the radial mycelial growth of the pathogens was observed for media containing 25 and 50% of the filtered growth medium from the cultivation of the fungus Fusarium solani Fs-K. In addition, measurements of the mycelial dry weight of the pathogen Fusarium oxysporum f.sp. radicis-lycopersici showed 30% decrease in dry biomass in the case that 50% culture filtrate was added in the medium compared to the respective control (medium containing 50% PDB and the amount of agar needed)

This indicates that the fungus of the present invention produces extracellular compounds which can inhibit the radial growth of phytopathogenetic filamentous fungi and oomycetes.
6. Enhancement of suppressive properties of composts against soil-borne phytopathogenic fungi by inoculation with the antagonistic fungus Fusarium solani Fs-K

Composts, produced in the Institute of Kalamata (N.AG.RE.F.-Greece), were used as substrates for the growth of tomato plants (line ACE55). These composts were produced from agricultural residues/by products (e.g. olive tree leaves, olive mill wastewaters, olive press-cake, extracted olive press-cake, spent mushroom substrate and grape marc). Plants were inoculated with the antagonistic fungus described in the present invention at concentration of 10⁵ conidia per cm³ of growth substrate, according to the standard procedure described in section 2. Plants were then infected by the pathogenic fungus Fusarium oxysporum f.sp. radicis-lycopersici and disease development was determined based on the bioassay described in section 2.

From the above experimental procedures, it was found that the antagonistic fungus of the present invention can be added in non-suppressive composts, in which it may grow and develop suppressiveness against plant pathogens

TABLE 1
Effect of inoculum concentration (conidia per cm³ of growth substrate) of
antagonistic fungus Fusarium solani Fs-K on the control of disease
incidence caused by F. oxysporum f.sp. radicis-lycopersici (FORL).

| | | | F. | | | | |
|---|---|---|---|---|---|---|---|
| | | | oxysporum | | | | |
| | | | f.sp. radicis- | | | | |
| | | | lycopersici | Fs-K:pathogen ratio^{II} | | | |
| Fungi | None | Fs-K | (FORL) | 1:10³ | 1:10² | 1:10 | 1:1 |
| % Disease | 0 | 0 | 88c | 52 b | 40 b | 22.6 a | 18.6 a |
| incidence^{I} | | | | | | | |

^{I}Disease incidence was assessed as percentage of dead plants (n = 25 or 30 plants). The antagonist and the pathogen were applied as inocula to the root system. Strain Fs-K was applied one week after sowing; FORL was applied at the stage of first-true-leaf.
^{II}FORL in the form of 10⁵ conidia cm⁻³ peat; strain Fs-K in the form of 10², 10³, 10⁴ and 10⁵ conidia cm⁻³ peat. Statistical comparisons between treatments were performed by Duncan's tests (a <0.05). Letters in common indicate a lack of significant difference.

1. A pure culture of the fungus Fusarium solani strain Fs-K, which consists of hyphae, conidia and chlamydospores of the fungus.

2. A process for preventing infection and/or protecting plants from infection by phytopathogenic microorganisms (fungi, bacteria) and nematodes, comprising inoculating with a culture of fungal strain Fusarium solani Fs-K before, during or after plant seeding.

3. A process according to claim 2, which uses the culture of the fungal strain Fusarium solani Fs-K as well as any extracts or produced molecules with inhibitory action against phytopathogenic organisms and which can be used on their own or in combination with other biological and chemical factors or following the genetic manipulation of the strain, resulting in the prevention of damage by pathogens and pests on plants or their products and in the regulation/modulation of plant rhizosphere.

4. A process according to claim 2, wherein the fungal strain Fusarium solani Fs-K of claim 1 is used for the biological control of the phytopathogenic fungus Fusarium oxysporum f.sp. radicis-lycopersici.

5. A process according to claim 2, wherein the fungal strain Fusarium solani Fs-K is used for the biological control of the phytopathogenic oomycete Phytophthora nicotianae.

6. A process according to claim 2, wherein the fungal strain Fusarium solani Fs-K is used for the biological control of the phytopathogenic fungus Septoria lycopersici.

7. A phytoprotective method against phytopathogenic microorganisms and nematodes according to claim 2, wherein the fungal strain Fusarium solani Fs-K is added in plant growth substrates.

8. A method according to claim 7, wherein the plant growth substrate is peat and/or mixtures with peat.

9. A method according to claim 7, wherein the plant growth substrate is compost (that is any product of aerobic thermophilic fermentation of agricultural, municipal or industrial wastes, by-products and/or mixtures of them).

10. A method according to claim 7, wherein the plant growth substrate is any inert material and/or mixtures of inert material.

11. A method according to claim 9, wherein the compost is derived by agro-industrial wastes or by-products.

12. A method according to claim 9, wherein the compost is derived from municipal wastes.

13. A method according to claim 6, wherein the plant growth substrate is peat or any other inert material (perlite or vermiculite or other material used in plant propagation) and compost.

14. A pure culture according to claim 1, wherein the fungal strain has been genetically modified for the improvement of its use.

15. A method according to claim 2, wherein the fungal strain Fusarium solani Fs-K is used for the production of inoculated plantlets, which could be transferred and/or transplanted to other sites for cultivation.

16. A method according to claim 2, wherein the plantlets are inoculated with conidia or hyphae or any other vegetative or propagative form of the fungal strain or with any combination of them.


SEQUENCE LISTING (4 sequences)

SEQ ID NO: 1 - DNA, 743 bp, Fusarium solani
  Strain FsK
ataggacgtgtggttctattttgttggtttctaggaccgccgtaatgattaatagggacagtcgggggcatcagtattcaattgtcagaggtgaaatycttggatttattgaagactaactactgcgaaagcatttgccaaggatgttttcattaatcaggaacgaaagttaggggatcgaagacgatcagataccgtcgtagtcttaaccataaactatgccgactagggatcggacggtgttatattttgacccgttcggcaccttacgagaaatcaaagtgcttgggctccagggggagtatggtcgcaaggctgaaacttaaagaaattgacggaagggcaccaccaggggtggagctgcggcttaatttgactcaacacggggaaactcaccaggtccagacacaatgaggattgacagattgagagctctttcttgattttgtgggtggtggtgcatggccgttcttagttggtggagtgatttgtctgcttaattgcgataacgaacgagaccttaacctgctaaatagcccgtattgctttggcagtacgctggcttcttagagggactatcggctcaagccgatggaagtttgaggcaataacaggtctgtgatgcccttagatgttctgggccgcacgcgcgctacactgacggagccagcgagtacttccttggccgaaaggcccgggtaatcttgttaaactccgtcgtgctggggatagrgcattgcaat

SEQ ID NO: 2 - DNA, 526 bp, Fusarium solani
  Strain FsK
gagggatcattaccgagttatacaactcatcaaccctgtgaacatacctataacgttgcctcggcgggaacagacggccccgtaacacgggccgcccccgccagaggaccccctaactctgtttctataatgtttcttctgagtaaacaagcaaataaattaaaactttcaacaacggatctcttggctctggcatcgatgaagaacgcagcgaaatgcgataagtaatgtgaattgcagaattcagtgaatcatcgaatctttgaacgcacattgcgcccgccagtattctggcgggcatgcctgttcgagcgtcattacaaccctcaggcccccgggcctggcgttggggatcggcggaagccccctgcgggcacaacgccgtcccccaaatacagtggcggtcccgccgcagcttccattgcgtagtagctaacacctcgcaactggagagcggcgcggccacgccgtaaaacacccaacttctgaatgttgacctcgaatcaggtaggaatacccgctgaac

SEQ ID NO: 3 - DNA, 19 bp, Artificial Sequence
  Description of Artificial Sequence Synthetic primer
tccgtaggtgaacctgcgg

SEQ ID NO: 4 - DNA, 20 bp, Artificial Sequence
  Description of Artificial Sequence Synthetic primer
tcctccgcttattgatatgc